(19) United States
(12) Patent Application Publication
(10) Pub. No.: US 2019/0169157 A1
(43) Pub. Date: Jun. 6, 2019

(54) HIGH REFRACTIVE INDEX LIQUIDS BASED ON META-SUBSTITUTED S-ALKYL THIOETHERS

(71) Applicant: BASF SE, Ludwigshafen am Rhein (DE)

(72) Inventors: Reinhold OEHRLEIN, Rheinfelden-Herten (DE); Jan BLOCH, Oberrohrdorf (CH); Gabriele BAISCH, Binzen (DE); Natalia KUKALEVA, Zurich (CH)

(73) Assignee: BASF SE, Ludwigshafen am Rhein (DE)

(21) Appl. No.: 16/324,751

(22) PCT Filed: Aug. 11, 2017
(86) PCT No.: PCT/EP2017/070502

§ 371 Date: Feb. 11, 2019

(30) Foreign Application Priority Data
Aug. 16, 2016 (EP) 16184278.6

(51) Int. Cl. C07D 339/06; C07C 319/18; C07C 323/52; C07F 9/40; C07C 321/28; C07C 319/14; G02B 1/04

(52) U.S. Cl. CPC C07D 339/06; C07C 319/18; C07C 323/52; C07F 9/4006; C07C 2601/16; C07C 319/14; G02B 1/04; C07C 2601/14; C07C 321/28

(57) ABSTRACT

The present invention relates to liquid aromatic thioethers, a process for the preparation of the liquid aromatic thioethers, an article comprising the liquid aromatic thioethers as well as the use of the liquid aromatic thioethers as a component or substantial part of an optical liquid, window material, color filter, coating, varnish, lacquer, dye or pigment formulation, immersion liquid, calibration liquid or matching liquid, ingredient or additive in a plastic material or ingredient or additive in a polymer.

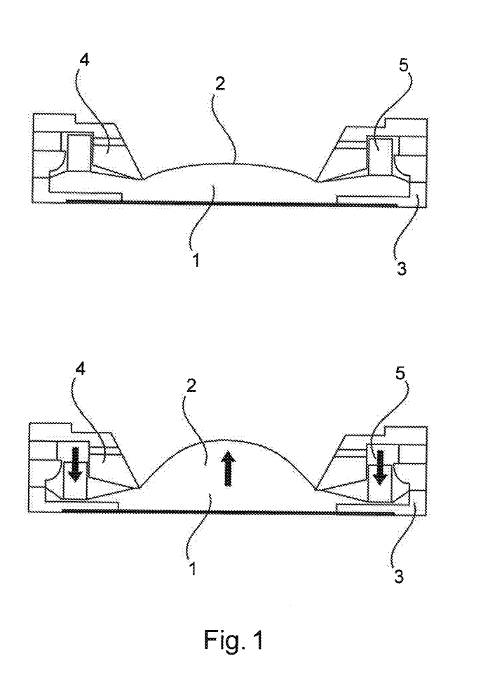

### FIELD OF THE INVENTION

The present invention relates to liquid aromatic thioethers, a process for the preparation of the liquid aromatic thioethers, an article comprising the liquid aromatic thioethers as well as the use of the liquid aromatic thioethers as a component or substantial part of an optical liquid, window material, color filter, coating, varnish, lacquer, dye or pigment formulation, immersion liquid, calibration liquid or matching liquid, ingredient or additive in a plastic material or ingredient or additive in a polymer.

### BACKGROUND OF THE INVENTION

The present application deals with the preparation and use of liquids with high refractive indices in high purity. The current development of ever smaller and lighter optical systems demands novel approaches for the technical realization of optical devices for these intended applications. Especially heavy and slow optical devices based on glass and/or solid lens systems which are moved back and forth to focus or zoom are sought to be replaced by so called liquid tunable lens systems. A crucial part of Optotune tuneable focus lens is a liquid-filled core element comprised of a rigid container and a deformable membrane, which enclose a so-called ‘optical liquid’. The lens is formed by a lens shaper, which defines the optically clear aperture of the lens and the initial deformation of the lens. In case of manually tuneable lenses (ML), the shaper is pushed directly into the membrane by hand/manually, whereas in case of electrically tuneable lenses (EL), an electromagnetically actuated bobbin is pushed into the membrane, deforming the liquid filled volume and therefore changing the curvature of the lens as a function of the applied force to the actuated bobbin as it is shown in Scheme 1 below.

A liquid is enclosed by a soft membrane and a rigid container. The initial shape of the lens is defined by the lens shaper and the amount of liquid in the volume. When a bobbin is pushed into the deformable membrane, fluid is pumped from the surrounding of the lens into the centre, resulting in a deformation of the central part of the lens and therefore in a change of the focal length of the lens

The liquid is a crucial component of this system. It is desirable to have liquids with high refractive index (≥1.5), with Abbe's number≥40, high transparency in the visible range (400-800 nm) of at least 90% transparency, low volatility, wide operational range of temperature from desirably about −20° C. to +100° C. and chemical compatibility with the other components of the complete lens device (e. g. membranes, container materials, glues). The viscosity of those liquids should be in the range of 100 mPas-5000 mPas.

Materials having both high Abbe's numbers ν (ν=(n_{D}−1)/n_{F}−n_{C}) with refractive indices n_{D} at 589.3 nm, n_{F} at 486.2 nm, n_{C} at 656.3 nm) and high refractive indices n ((n=sin αᵢ/sin αᵣ), i meaning angle of incident light and r angle of reflective light) are not easy to obtain because both features normally cannot be enhanced in parallel and must be balanced.

There are only a limited number of materials known in the literature fulfilling some of the above mentioned requirements. Commercially available fluorinated siloxanes (GE-LEST) show e.g. Abbe's numbers of ν>100 but have very low refractive indices n<1.3. Recently, a new class of polythio ether sulfones based compounds exhibiting high Abbe's numbers and high refractive indices have been reported by Y. Suzuki et al. Macromolecules, 2012, 45, 3402 (compare scheme 2). However, all those materials are solids at room temperature including their monomeric building blocks and are therefore not acceptable for the claimed use.

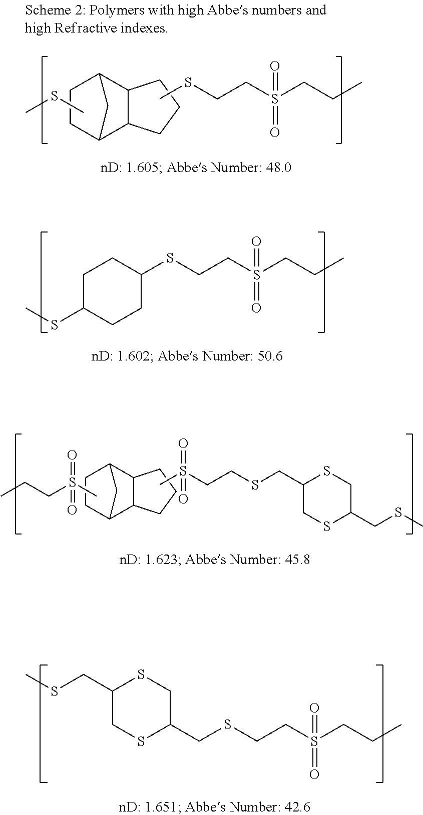

Furthermore, WO 2016/046292 A1 refers to materials consisting of poly-alkyl thioethers derived from the Michael-addition of alkylthiols to acrylic acid ester derivatives (selected examples in Scheme 3).

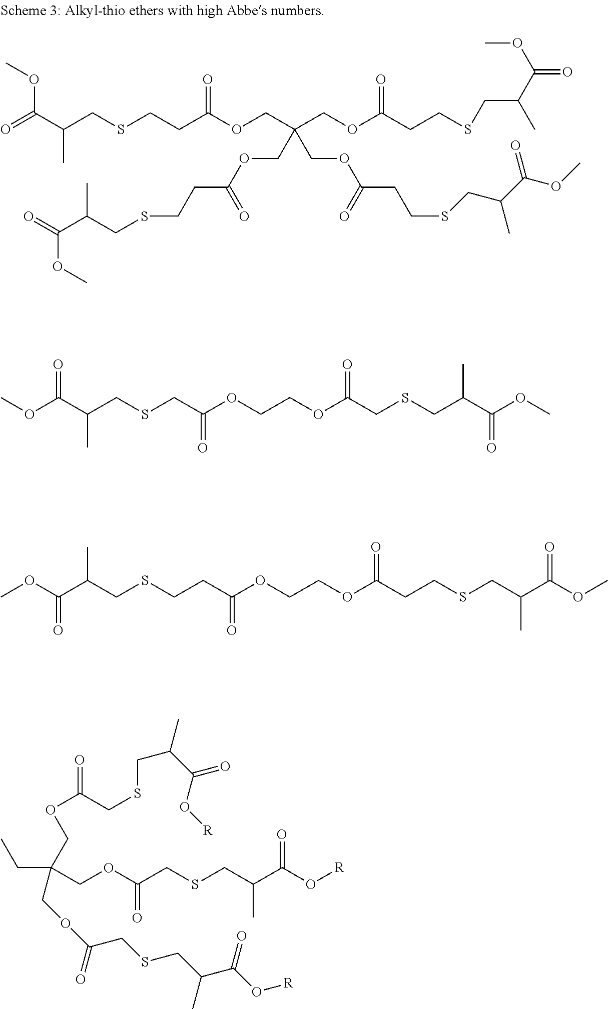

Although, these compounds fulfill a number of the desired physical parameters (e.g exceedingly high Abbe's number), the refractive indices need to be further improved. As their miscibility with other liquids is limited, there is still a need for more advanced compounds.

Still another class of polymeric materials has been disclosed by T. Okubo et al. J. Appl. Polmer Sci., 1998, 68, 1791 and T. Okubo et al. J. Mater Sci., 1999, 34, 337. Currently, optical fluids based on the thiophenol ether motif (n >1.6) are considered as ingredients (scheme 4).

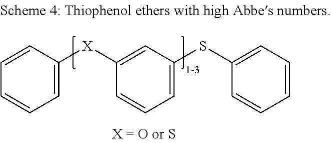

These compounds are, however, only available as undefined mixtures of complex composition and are very tedious to prepare because of the concomitant direct linkage of a sulfur atom to two aromatics. Therefore highly precise applications in the said devices cannot be reproduced reliably with these materials. In addition the quality is variable and available amounts are low.

Other materials containing the oligo-sulfide motif and additionally reactive acrylic residues are used to produce polymers with high n_{D} and ν (e.g. DE 4011686, EP284374). However, these materials are not liquid and thus are not suitable for the intended use.

Therefore, there is still a need in the art for providing compounds having a high refractive index n as well as high Abbe's number u. In particular, it is desirable to provide compounds which are suitable for use in lens systems, and especially as optical liquids. Furthermore, it is desirable to provide compounds which are easy to access and are miscible with other liquid ingredients of the lens system and are compatible with the other device components.

Accordingly, it is an object of the present invention to provide compounds which are suitable for the use in lens systems, and especially as optical liquids. Furthermore, it is an object of the present invention to provide compounds having a high refractive index n as well as high Abbe's number u. A still further object of the present invention is to provide compounds which are easy to access. An even further object of the present invention is to provide compounds being miscible with other liquid ingredients of the lens system and are compatible with the other device components. Further objects can be gathered from the following description of the invention.

### SUMMARY OF THE INVENTION

The foregoing and other objects are solved by the subject-matter of the present invention. According to a first aspect of the present invention, liquid aromatic thioethers are provided. The liquid aromatic thioether is a compound of the formula A1

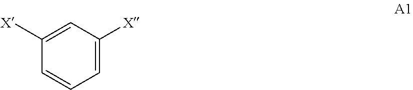

wherein X′=Cl, Br or I and X″=S(CH₂CHR1)-Z with Z=radical C(O)G, radical C(O)A-G or radical P(O)(OR2)₂, G=H, Me, Et, ⁱProp, ⁿProp or ^{t}But, A=O or NH, R1=H or Me and R2=Me, Et, ⁿProp or ⁱProp or X′=X″.

In one embodiment, the liquid aromatic thioether of formula A1 is a reaction product of compounds of the formulae A and B

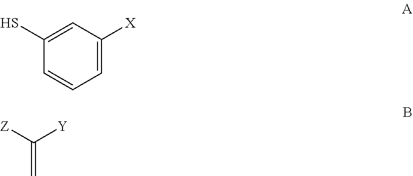

wherein in formula A X=Cl, Br, I or SH; and in formula B Y=H, Me, Et, ⁱProp, ⁿProp or ^{t}But, Z=radical C(O)G, radical C(O)A-G or radical P(O)(OR2)₂ with G=H, Me, Et, ⁱProp, ⁿProp or ^{t}But, A=O or NH and R2=Me, Et, ⁿProp or ⁱProp.

According to another aspect, the liquid aromatic thioether is a compound of the formula A2

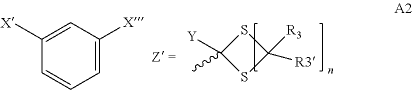

wherein X′=Cl, Br or I and X′″=S(CH₂CHR1)-Z′ with Z′ as structurally defined above with R1, R3 and R3′=independently H or Me, n=2 or 3 and Y=H, Me, Et, ⁱProp, ⁿProp or ^{t}But or X′=X′″.

In one embodiment, the liquid aromatic thioether of formula A2 is a reaction product of compounds of the formulae A1 and C

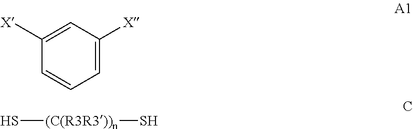

wherein in formula A1 X′=Cl, Br or I and X″=S(CH₂CHR1)-Z with Z=radical C(0)G with G=H, Me, Et, ⁱProp, ⁿProp or ^{t}But, and R1=H or Me, or X′=X″; and in formula C n=2 or 3 and R3 and R3′=independently H or Me.

According to a further aspect, the liquid aromatic thioether is a compound of the formula A3

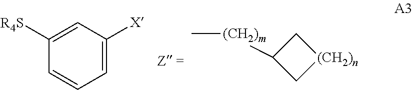

wherein X′=I or Br and R4=C1 to C8 linear or branched alkyl chain, a carbocycle with 5 or 6 C-atoms or Z″ as structurally defined above and n and m=independently 1, 2 or 3 and where one or two non-neighbouring ring-forming —CH₂-moieties of Z″ are replaced by sulfur.

According to one embodiment, the liquid aromatic thioether of formula A3 is a reaction product of a compound of the formula D and an alkylthiol HSR4

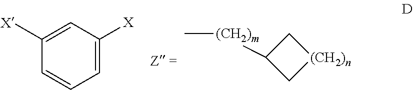

Wherein in compound D X=I and X′=Br or X=X′=I or Br and in the alkylthiol R4=C1 to C8 linear or branched alkyl chain, a carbocycle with 5 or 6 C-atoms or Z″ as structurally defined above with n and m=independently 1, 2 or 3 and where one or two non-neighbouring ring-forming —CH₂-moieties of Z″ are replaced by sulfur.

According to a still further aspect, the liquid aromatic thioether is a compound of the formula A4

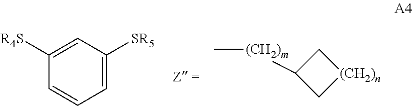

wherein R4 and R5=independently C1 to C8 linear or branched alkyl chain, a carbo-cycle with 5 or 6 C-atoms or Z″ as structurally defined above with m and n=independently 1, 2 or 3 and where one or two non-neighbouring ring-forming —CH₂-moieties of Z″ are replaced by sulfur and wherein R4 and R5 are the same or different.

According to one embodiment, the liquid aromatic thioether of formula A4 is a reaction product of a compound of the formula D and an alkylthiol HSR5

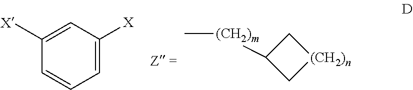

wherein in formula D X=I and X′=Br or X=X′=I or Br and in the alkylthiol HSR5 R5=C1 to C8 linear or branched alkyl chain, a carbocycle with 5 or 6 C-atoms or Z″ as structurally defined above with m and n=independently 1, 2 or 3 and where one or two non-neighbouring ring-forming —CH₂-moieties of Z″ are replaced by sulfur, wherein R4 and R5 are the same or different.

According to another embodiment, the liquid aromatic thioether of formula A4 is a reaction product of a compound of formula A3 with Mg to form a Grignard reagent of formula A3

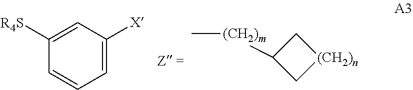

wherein in formula A3 X′=I or Br and R4=C1 to C8 linear or branched alkyl chain, a carbocycle with 5 or 6 C-atoms or Z″ as structurally defined above with m and n=independently 1, 2 or 3 and where one or two non-neighbouring ring-forming —CH₂-moieties of Z″ are replaced by sulfur; and further reacting the Grignard reagent of A3 with R5SSO₃M with M=Li or Na and R5=C1 to C8 linear or branched alkyl chain, a carbo-cycle with 5 or 6 C-atoms or Z″ as structurally defined above with m and n=independently 1, 2 or 3 and where one or two non-neighbouring ring-forming —CH₂-moieties of Z″ are replaced by sulfur, wherein R4 and R5 are the same or different.

According to one embodiment, the liquid aromatic thioether of the present invention has a refractive index in the range from 1.50 to 1.9 and/or an Abbe's number in the range from 25 to 110.

According to a further aspect of the present invention, a process for the preparation of a liquid aromatic thioether, as defined herein, is provided. The process comprising the steps of
A) providing the compounds of the formulae A and B as defined herein, or
B) providing the compounds of the formulae Al and C as defined herein, or
C) providing the compound of the formula D and an alkylthiol HSR4 as defined herein, or
D) providing the compound of the formula A3 and an alkylthiol HSR5 or the compound of the formula A3 and Mg and R5SSO₃M as defined herein; and
E) reacting the compounds provided in step A) or B) or C) or D) such as to obtain the liquid aromatic thioether.

According to an even further aspect of the present invention, an article, preferably an optical lens such as a tuneable focus lens, optical liquid, wave guide material, tiltable prism, window material, color filter, coating, varnish, lacquer, dye or pigment formulation, immersion liquid, calibration liquid or matching liquid, ingredient or additive in a plastic material, ingredient or additive in a polymer, comprising the liquid aromatic thioether as defined herein as an ingredient in at least one of its parts or as part of the article itself is provided.

According to a still further aspect of the present invention, the use of the liquid aromatic thioether as defined herein as a component or substantial part of an optical liquid, window material, color filter, coating, varnish, lacquer, dye or pigment formulation, immersion liquid, calibration liquid or matching liquid, ingredient or additive in a plastic material or ingredient or additive in a polymer is provided.

Advantageous embodiments of the inventive liquid aromatic thioether are defined in the corresponding sub-claims.

In the following, the details and preferred embodiments of the inventive liquid aromatic thioethers will be described in more detail. It is to be understood that these technical details and embodiments also apply to the inventive process for the preparation of the inventive liquid aromatic thioethers, the inventive article comprising the liquid aromatic thioethers and its use.

### DETAILED DESCRIPTION OF THE INVENTION

It has been surprisingly found out that sulfur-rich meta-disubstituted thiophenols add equally well to electron-deficient C-C double bonds resulting in homogeneous, stable materials. Based on an alkylation and/or Michael-addition of these meta-disubstituted thio(dithio)phenols, S-alkylthiophenolethers result which are structurally well defined are obtained and further are easy to purify. Accordingly, the strict physico-optical requirements as stated above are significant. The meta-substituted motif of the aromatic system is also relevant for keeping the compounds fluid in the temperature range of interest. The inventive liquid aromatic thioethers also provide compatibility with membrane materials.

According to one aspect of the present invention, a liquid aromatic thioether is provided being a compound of the formula A1

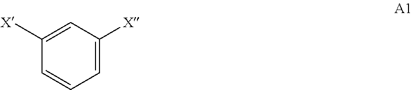

wherein X′=Cl, Br or I and X″=SCH₂(CHR1)-Z with Z=radical C(O)G, radical C(O)A-G or radical P(O)(OR2)₂, G=H, Me, Et, ⁱProp, ⁿProp or ^{t}But, A=O or NH, R1=H or Me and and R2=Me, Et, ⁿProp or ⁱProp or X′=X″.

Preferably, the liquid aromatic thioether is a compound of the formula A1, wherein X′=Br and X″=S(CH₂CHR1)-Z with Z=radical C(O)G, radical C(O)A-G or radical P(O)(OR2)₂, G=H, Me, Et, ⁱProp, ⁿProp or ^{t}But, A=O or NH, R1=H or Me and R2=Me, Et, ⁿProp or ⁱProp.

Alternatively, the liquid aromatic thioether is a compound of the formula A1, wherein X′=X″=S(CH₂CHR1)-Z with Z=radical C(O)G, radical C(O)A-G or radical P(O)(OR2)₂, G=H, Me, Et, ⁱProp, ⁿProp or ^{t}But, A=O or NH, R1=H or Me and R2=Me, Et, ⁿProp or ⁱProp.

The liquid aromatic thioether of the formula Al is preferably a reaction product of the meta-substituted thiophenol precursor A and olefin B,

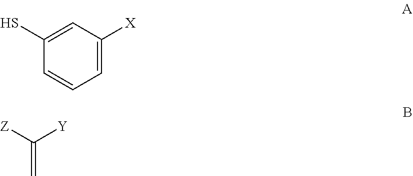

wherein in formula A X=Cl, Br, I or SH; and in formula B Y=H, Me, Et, ⁱProp, ⁿProp or ^{t}But, Z=radical C(O)G, radical C(O)A-G or radical P(O)(OR2)₂, with G=H, Me, Et, ⁱProp, ⁿProp or ^{t}But, A=O or NH and R2=Me, Et, ⁿProp or ⁱProp resulting in A1.

In one embodiment, the liquid aromatic thioether of the formula A1 is a reaction product of the meta-substituted thiophenol precursor A and olefin B, wherein in formula A X=Cl, Br or I; and in formula B Y=H, Me, Et, ⁱProp or ⁿProp, Z=radical C(O)G, radical C(O)A-G or radical P(O)(OR2)₂, G=H, Me, Et, ⁱProp, ⁿProp or ^{t}But, A=O or NH and R2=Me, Et, ⁿProp or ⁱProp. In this embodiment, the obtained liquid aromatic thioether obtained is preferably of the formula A1, wherein X′=Cl, Br or I and X″=S(CH₂CHR1)-Z with Z=radical C(O)G, radical C(O)A-G or radical P(O)(OR2)₂, G=H, Me, Et, ⁱProp, ⁿProp or ^{t}But, A=O or NH, R1=H or Me and R2=Me, Et, ⁿProp or ⁱProp.

For example, the liquid aromatic thioether of the formula A1 is a reaction product of the meta-substituted thiophenol precursor A and olefin B, wherein in formula A X=Br; and in formula B Y=H or Me, Z=radical C(O)A-G with G=Me or ^{t}But, A=O. In this embodiment, the obtained liquid aromatic thioether obtained is preferably of the formula A1, wherein X′=Br and X″=S(CH₂CHR1)-Z with Z=radical C(O)A-G with G=Me or ^{t}But, A=O and R1=H or Me.

For example, the liquid aromatic thioether of the formula A1 is a reaction product of the meta-substituted thiophenol precursor A and olefin B, wherein in formula A X=Br; and in formula B Y=H or Me, Z=radical P(O)(OR2)₂, with R2=Et. In this embodiment, the obtained liquid aromatic thioether obtained is preferably of the formula A1, wherein X′=Br and X″=S(CH₂CHR1)-Z with Z=radical P(O)(OR2)₂, R1=H or Me and R2=Et.

Alternatively, the liquid aromatic thioether of the formula A1 is a reaction product of the meta-substituted thiophenol precursor A and olefin B, wherein in formula A X=SH; and in formula B Y=H, Me, Et, ⁱProp or ⁿProp, Z=radical C(O)G, radical C(O)A-G or radical P(O)(OR2)₂, G=H, Me, Et, ⁱProp, ⁿProp or ^{t}But, A=O or NH, R2=Me, Et, ⁿProp or ⁱProp. In this embodiment, the obtained liquid aromatic thioether obtained is preferably of the formula A1, wherein X′=X″=S(CH₂CHR1)-Z with Z=radical C(O)G, radical C(O)A-G or radical P(O)(OR2)₂, G=H, Me, Et, ⁱProp, ⁿProp or ^{t}But, A=O or NH, R1=H or Me and R2=Me, Et, ⁿProp or ⁱProp.

For example, the liquid aromatic thioether of the formula A1 is a reaction product of the meta-substituted thiophenol precursor A and olefin B, wherein in formula A X=SH; and in formula B Y=H or Me, Z=radical C(O)A-G with G=Me or ^{t}But and A=O. In this embodiment, the obtained liquid aromatic thioether obtained is preferably of the formula A1, wherein X′=X″=S(CH₂CHR1)-Z with Z=radical C(O)A-G, G=Me or ^{t}But, A=O and R1=H or Me.

For example, the liquid aromatic thioether of the formula A1 is a reaction product of the meta-substituted thiophenol precursor A and olefin B, wherein in formula A X=SH; and in formula B Y=H or Me, Z=radical P(O)(OR2)₂ with R2=Me or Et. In this embodiment, the obtained liquid aromatic thioether obtained is preferably of the formula A1, wherein X′=X″=S(CH₂CHR1)-Z with Z=radical P(O)(OR2)₂, R1=H or Me and R2=Me or Et.

Further when Z=C(O)G with G=H, Me, Et, ⁱProp, ⁿProp or ^{t}But in formula A1, A1 is preferably reacted with a compound C with n=2 or 3 and R3 and R3′ independently=H, Me or Et resulting in a compound of formula A2.

Thus, according to another aspect, the liquid aromatic thioether is a compound of the formula A2

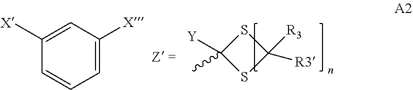

wherein X′=Cl, Br or I and X″′=S(CH₂CHR1)-Z′ with Z′ as structurally defined above with R1, R3 and R3′=independently H or Me, n=2 or 3 and Y=H, Me, Et, ⁱProp, ⁿProp or ^{t}But or X′=X′″.

For example, the liquid aromatic thioether is a compound of the formula A2, wherein X′=Cl, Br or I and X″′=S(CH₂CHR1)-Z′ with Z′ as structurally defined above with R1, R3 and R3′=independently H or Me, n=2 or 3 and Y=H, Me, Et, ⁱProp, ⁿProp or ^{t}But.

Alternatively, the liquid aromatic thioether is a compound of the formula A2, wherein X′=X′″=S(CH₂CHR1)-Z′ with Z′ as structurally defined above with R1=H or Me, and R3 and R3′=independently H or Me, n=2 or 3 and Y=H, Me, Et, ⁱProp, ⁿProp or ^{t}But.

The liquid aromatic thioether of the formula A2 is thus preferably a reaction product of the meta-substituted thiophenol precursor A1 and compound C

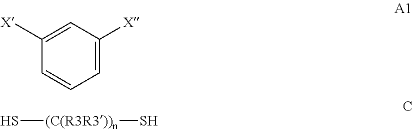

wherein in formula A1 X′=Cl, Br or I and X″=S(CH₂CHR1)-Z with Z=radical C(O)G; G=H, Me, Et, ⁱProp, ⁿProp or ^{t}But, and R1=H or Me or X′=X″; and in formula C n=2 or 3 and R3 and R3′=independently H or Me.

In one embodiment, the liquid aromatic thioether of the formula A2 is a reaction product of the meta-substituted thiophenol precursor A1 and compound C, wherein in formula A1 X′=Br and X″=S(CH₂CHR1)-Z with Z=radical C(O)G, G=H and R1 is H or Me; and in formula C n=2 and R3 and R3′=H or Me. In this embodiment, the liquid aromatic thioether is a compound of the formula A2, wherein X′=Br and X″′=S(CH₂CHR1)-Z′ with Z′ as structurally defined above with R1, R3 and R3′=independently H or Me, n=2 and Y =H or Me.

For example, the liquid aromatic thioether of the formula A2 is a reaction product of the meta-substituted thiophenol precursor A1 and compound C, wherein in formula A1 X′=Br and X″=S(CH₂CHR1)-Z with Z =radical C(0)G, G =H and R1 =H or Me; and in formula C n=2 and R3 and R3′=H . In this embodiment, the liquid aromatic thioether is a compound of the formula A2, wherein X′=Br and X″′=S(CH₂CHR1)-Z′ with Z′ as structurally defined above with R1=H or Me and R3 and R3′=H, n=2 and Y=H.

For example, the liquid aromatic thioether of the formula A2 is a reaction product of the meta-substituted thiophenol precursor A1 and compound C, wherein in formula A1 X′=Br and X″=S(CH₂CHR1)-Z with Z=radical C(O)G, G=H and R1=H or Me; and in formula C n=2 and R3 and R3′=H. In this embodiment, the liquid aromatic thioether is a compound of the formula A2, wherein X′=Br and X″′=S(CH₂CHR1)-Z′ with Z′ as structurally defined above with R1=H or Me and R3 and R3′=H, n=2 and Y=Me.

Alternatively, the liquid aromatic thioether of the formula A2 is a reaction product of the meta-substituted thiophenol precursor Al and compound C, wherein in formula A1 X′=X″=S(CH₂CHR1)-Z with Z=radical C(O)G, G=H and R1=H or Me; and in formula C n=2 and R3 and R3′=H or Me. In this embodiment, the liquid aromatic thioether is a compound of the formula A2, wherein X′=X′″=S(CH₂CHR1)-Z′ with Z′ as structurally defined above with R1, R3 and R3′=independently H or Me, n=2 and Y=H or Me.

For example, the liquid aromatic thioether of the formula A2 is a reaction product of the meta-substituted thiophenol precursor A1 and compound C, wherein in formula A1 X′=X″=S(CH₂CHR1)-Z with Z=radical C(O)G, G=H and R1=H or Me; and in formula C n=2 and R3 and R3′=H. In this embodiment, the liquid aromatic thioether is a compound of the formula A2, wherein X′=X′″=S(CH₂CHR1)-Z′ with Z′ as structurally defined above with R1=H or Me and R3 and R3′=H, n=2 and Y=H.

For example, the liquid aromatic thioether of the formula A2 is a reaction product of the meta-substituted thiophenol precursor A1 and compound C, wherein in formula A1 X′=X″=S(CH₂CHR1)-Z with Z=radical C(O)G, G=H and R1=H or Me; and in formula C n=2 and R3 and R3′=H. In this embodiment, the liquid aromatic thioether is a compound of the formula A2, wherein X′=X′″=S(CH₂CHR1)-Z′ with Z′ as structurally defined above with R1=H or Me and R3 and R3′=H, n=2 and Y=Me.

According to another aspect of the present invention, the liquid aromatic thioether is a compound of the formula A3

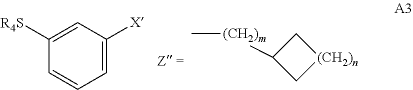

wherein X′=I or Br and R4=C1 to C8 linear or branched alkyl chain, a carbocycle with 5 or 6 C-atoms or Z″ as structurally defined above with n and m=independently 1, 2 or 3 and where one or two non-neighbouring ring-forming —CH2-moieties of Z″ are replaced by sulfur.

The liquid aromatic thioether of the formula A3 is preferably a reaction product of a compound of the formula D and an alkylthiol HSR4

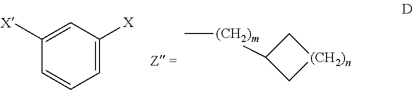

wherein in compound D X=I and X′=Br or X=X′=I or Br and in the alkylthiol R4=C1 to C8 linear or branched alkyl chain, a carbocycle with 5 or 6 C-atoms or Z″ as structurally defined above with n and m=independently 1, 2 or 3 and where one or two non-neighbouring ring-forming —CH₂-moieties of Z″ are replaced by sulfur.

For example, the liquid aromatic thioether of the formula A3 is preferably a reaction product of a compound of the formula D and an alkylthiol HSR4, wherein in compound D X=I and X′=Br and in the alkylthiol R4=C1 to C8 linear or branched alkyl chain, preferably a C4 to C8 branched alkyl chain, more preferably, a C5 or C6 branched alkyl chain and most preferably a C5 branched alkyl chain. In this embodiment, the liquid aromatic thioether is a compound of the formula A3, wherein X′=Br and R4=C1 to C8 linear or branched alkyl chain, preferably a C4 to C8 branched alkyl chain, more preferably, a C5 or C6 branched alkyl chain and most preferably a C5 branched alkyl chain. In another embodiment, the liquid aromatic thioether of the formula A3 is preferably a reaction product of a compound of the formula D and an alkylthiol HSR4, wherein in compound D X=I and X′=Br and in the alkylthiol R4=a carbocycle with 5 or 6 C-atoms, preferably 6 C-atoms. In this embodiment, the liquid aromatic thioether is a compound of the formula A3, wherein X′=Br and R4=a carbocycle with 5 or 6 C-atoms, preferably 6 C-atoms.

In another embodiment, the liquid aromatic thioether of the formula A3 is preferably a reaction product of a compound of the formula D and an alkylthiol HSR4, wherein in compound D X=I and X′=Br and in the alkylthiol R4=Z″ as structurally defined above with n and m=independently 1 or 2 and where one or two, preferably two, non-neighbouring ring-forming —CH₂-moieties of Z″ are replaced by sulfur. In this embodiment, the liquid aromatic thioether is a compound of the formula A3, wherein X′=Br and R4=Z″ as structurally defined above with n and m=independently 1 or 2 and where one or two, preferably two, non-neighbouring ring-forming —CH₂-moieties of Z″ are replaced by sulfur.

If R4=Z″ as structurally defined above, it is appreciated that preferably two non-neighbouring ring-forming —CH2-moieties of Z″ are replaced by sulfur. It is even more preferred that one of the two sulfur moieties is in the ortho-position with regard to the chemical bond built by the reaction to the compound D.

It is preferred that the liquid aromatic thioether of the formula A3 is preferably obtained by a Cu⁺ promoted substitution reaction according to F. Y. Kwong et al. Org. Lett. 2002, 4, 3517, which is thus incorporated herewith by reference.

According to another aspect of the present invention, the liquid aromatic thioether is a compound of the formula A4

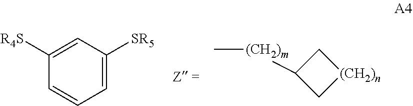

wherein R4 and R5=independently C1 to C8 linear or branched alkyl chain, a carbo-cycle with 5 or 6 C-atoms or Z″ as structurally defined above with m and n=independently 1, 2 or 3 and where one or two non-neighbouring ring-forming —CH₂-moieties of Z″ are replaced by sulfur and wherein R4 and R5 are the same or different.

The liquid aromatic thioether of the formula A4 is preferably a reaction product of a compound of the formula D and an alkylthiol HSR5,

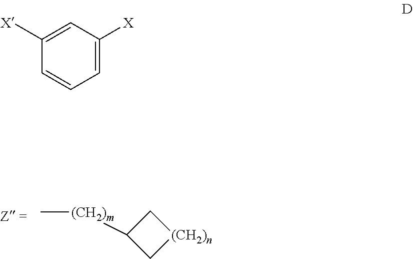

wherein in formula D X=I and X′=Br or X=X′=I or Br, and in the alkylthiol HSR5, R5=C1 to C8 linear or branched alkyl chain, a carbocycle with 5 or 6 C-atoms or Z″ as structurally defined above with m, n=1-3 and where one or two non-neighbouring ring-forming —CH₂-moieties of Z″ are replaced by sulfur, wherein R4 and R5 are the same or different.

For example, the liquid aromatic thioether of the formula A4 is a reaction product of a compound of the formula D and an alkylthiol HSR5, wherein in formula D X=X′=I or Br; and in the alkylthiol HSR5, R5=C1 to C8 linear or branched alkyl chain, preferably a C4 to C8 branched alkyl chain, more preferably, a C5 or C6 branched alkyl chain and most preferably a C5 branched alkyl chain; and wherein R4 and R5 are the same. In this embodiment, the liquid aromatic thioether is a compound of the formula A4, wherein R4 and R5=C1 to C8 linear or branched alkyl chain, preferably a C4 to C8 branched alkyl chain, more preferably, a C5 or C6 branched alkyl chain and most preferably a C5 branched alkyl chain; and wherein R4 and R5 are the same.

In an alternative embodiment, the liquid aromatic thioether of the formula A4 is a reaction product of a compound of the formula D and an alkylthiol HSR5, wherein in formula D, X=X′=I or Br; and in the alkylthiol HSR5, R5=a carbocycle with 5 or 6 C-atoms, preferably 6 C-atoms; and wherein R4 and R5 are the same. In this embodiment, the liquid aromatic thioether is a compound of the formula A4, wherein R4 and R5=a carbocycle with 5 or 6 C-atoms, preferably 6 C-atoms; and wherein R4 and R5 are the same.

In an alternative embodiment, the liquid aromatic thioether of the formula A4 is a reaction product of a compound of the formula D and an alkylthiol HSR5, wherein in formula D X=X′=I or Br; and in the alkylthiol HSR5, R5=C1 to C8 linear or branched alkyl chain, preferably a C4 to C8 branched alkyl chain, more preferably, a C5 or C6 branched alkyl chain and most preferably a C5 branched alkyl chain; and wherein R4 and R5 are different. In this embodiment, the liquid aromatic thioether is a compound of the formula A4, wherein R4=a carbocycle with 5 or 6 C-atoms, preferably 6 C-atoms and R5=C1 to C8 linear or branched alkyl chain, preferably a C4 to C8 branched alkyl chain, more preferably, a C5 or C6 branched alkyl chain and most preferably a C5 branched alkyl chain; and wherein R4 and R5 are different.

In an alternative embodiment, the liquid aromatic thioether of the formula A4 is a reaction product of a compound of the formula D and an alkylthiol HSR5, wherein in formula D X=X′=I or Br; and in the alkylthiol HSR5, R5=Z″ as structurally defined above with m and n=independently 1 or 2 and where one or two, preferably two, non-neighbouring ring-forming —CH2-moieties of Z″ are replaced by sulfur; and wherein R4 and R5 are the same. In this embodiment, the liquid aromatic thioether is a compound of the formula A4, wherein R4 and R5=Z″ as structurally defined above with m and n=independently 1 or 2 and where one or two, preferably two, non-neighbouring ring-forming —CH2-moieties of Z″ are replaced by sulfur; and wherein R4 and R5 are the same.

It is preferred that the liquid aromatic thioether of the formula A4 obtained by the process as described above is preferably obtained by a Cu⁺ promoted substitution reaction according to F. Y. Kwong et al. Org. Lett. 2002, 4, 3517, which is thus incorporated herewith by reference.

Thus, if the liquid aromatic thioether of the formula A4 is a reaction product of a compound of the formula D and an alkylthiol HSR5, the liquid aromatic thioether of the formula A4 is preferably obtained by a Cu⁺ promoted substitution reaction.

Alternatively, the liquid aromatic thioether of the formula A4 may also be prepared from A3, whereby A3 is first treated with Mg in a dry ether solvent at or below 0° C. to form an intermediate Grignard reagent, which is scavenged in situ (at 0° C. to room temperature) with a Bunte salt, R5SSO₃M with M=Li, Na. The synthesis of Bunte salts is known from e.g. Houben_Weyl: Methoden der Org. Chemie, vol. 9, 1955, p. 67, which is thus incorporated by references.

The liquid aromatic thioether of the formula A4 can thus be a reaction product of a compound of formula A3 with Mg to form a Grignard reagent of formula A3

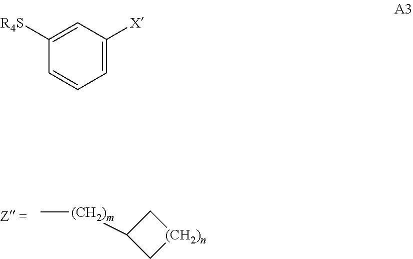

wherein in formula A3 X′=I or Br and R4=Cl to C8 linear or branched alkyl chain, a carbocycle with 5 or 6 C-atoms or Z″ as structurally defined above with m and n=independently 1, 2 or 3 and where one or two non-neighbouring ring-forming —CH2-moieties of Z″ are replaced by sulfur; and

further reacting the Grignard reagent of A3 with R5SSO₃M with M=Li or Na and R5=C1 to C8 linear or branched alkyl chain, a carbocycle with 5 or 6 C-atoms or Z″ as structurally defined above with m and n=independently 1, 2 or 3 and where one or two non-neighbouring ring-forming —CH₂-moieties of Z″ are replaced by sulfur, wherein R4 and R5 are the same or different.

For example, the liquid aromatic thioether of the formula A4 is a reaction product of a compound of formula A3 with Mg to form a Grignard reagent of formula A3, wherein in formula A3 X′=Br and R4=C1 to C8 linear or branched alkyl chain, preferably a C4 to C8 branched alkyl chain, more preferably, a C5 or C6 branched alkyl chain and most preferably a C5 branched alkyl chain; and

further reacting the Grignard reagent of A3 with R5SSO₃M with M=Li or Na and R5=C1 to C8 linear or branched alkyl chain, preferably a C4 to C8 branched alkyl chain, more preferably, a C5 or C6 branched alkyl chain and most preferably a C5 branched alkyl chain, wherein R4 and R5 are the same. In this embodiment, the liquid aromatic thioether is a compound of the formula A4, wherein R4 and R5=C1 to C8 linear or branched alkyl chain, preferably a C4 to C8 branched alkyl chain, more preferably, a C5 or C6 branched alkyl chain and most preferably a C5 branched alkyl chain; and wherein R4 and R5 are the same.

In an alternative embodiment, the liquid aromatic thioether of the formula A4 is a reaction product of a compound of formula A3 with Mg to form a Grignard reagent of formula A3, wherein in formula A3 X′=Br and R4=a carbocycle with 5 or 6 C-atoms, preferably 6 C-atoms; and

further reacting the Grignard reagent of A3 with R5SSO₃M with M=Li or Na and R5=a carbocycle with 5 or 6 C-atoms, preferably 6 C-atoms, wherein R4 and R5 are the same. In this embodiment, the liquid aromatic thioether is a compound of the formula A4, wherein R4 and R5=a carbocycle with 5 or 6 C-atoms, preferably 6 C-atoms; and wherein R4 and R5 are the same.

In an alternative embodiment, the liquid aromatic thioether of the formula A4 is a reaction product of a compound of formula A3 with Mg to form a Grignard reagent of formula A3, wherein in formula A3 X′=Br and R4=a carbocycle with 5 or 6 C-atoms, preferably 6 C-atoms; and

further reacting the Grignard reagent of A3 with R5SSO₃M with M=Li or Na and R5=C1 to C8 linear or branched alkyl chain, preferably a C4 to C8 branched alkyl chain, more preferably, a C5 or C6 branched alkyl chain and most preferably a C5 branched alkyl chain; and wherein R4 and R5 are different. In this embodiment, the liquid aromatic thioether is a compound of the formula A4, wherein R4 =a carbocycle with 5 or 6 C-atoms, preferably 6 C-atoms and R5=Cl to C8 linear or branched alkyl chain, preferably a C4 to C8 branched alkyl chain, more preferably, a C5 or C6 branched alkyl chain and most preferably a C5 branched alkyl chain; and wherein R4 and R5 are different.

In an alternative embodiment, the liquid aromatic thioether of the formula A4 is a reaction product of a compound of formula A3 with Mg to form a Grignard reagent of formula A3, wherein in formula A3 X′=I and R4=Z″ as structurally defined above with m and n=independently 1 or 2 and where one or two, preferably two, non-neighbouring ring-forming —CH₂-moieties of Z″ are replaced by sulfur; and

further reacting the Grignard reagent of A3 with R5SSO₃M with M=Li or Na and R5=Z″ as structurally defined above with m and n=independently 1 or 2 and where one or two, preferably two, non-neighbouring ring-forming —CH2-moieties of Z″ are replaced by sulfur, wherein R4 and R5 are the same. In this embodiment, the liquid aromatic thioether is a compound of the formula A4, wherein R4 and R5=Z″ as structurally defined above with m and n=independently 1 or 2 and where one or two, preferably two, non-neighbouring ring-forming —CH₂-moieties of Z″ are replaced by sulfur; and wherein R4 and R5 are the same.

It is appreciated that the liquid aromatic thioether of the present invention has specifically high Abbe's number u as well as high refractive index n.

For example, the liquid aromatic thioether has a refractive index in the range from 1.50 to 1.9 and/or an Abbe's number in the range from 25 to 110. Preferably, the liquid aromatic thioether has a refractive index in the range from 1.50 to 1.9 or an Abbe's number in the range from 25 to 110. Alternatively, the liquid aromatic thioether has a refractive index in the range from 1.50 to 1.9 or an Abbe's number in the range from 25 to 110. More preferably, the liquid aromatic thioether has a refractive index of 1.50 to 1.8 or an Abbe's number of 25 to 80. Alternatively, the liquid aromatic thioether has a refractive index of 1.5 to 1.8 and an Abbe's number of 25 to 80.

The present invention is further directed to a process for the preparation of a liquid aromatic thioether as defined herein, the process comprising the steps of
A) providing the compounds of the formulae A and B as defined herein, or
B) providing the compounds of the formulae A1 and C as defined herein, or
C) providing the compound of the formula D and an alkylthiol HSR4 as defined herein, or
D) providing the compound of the formula D and an alkylthiol HSR5 or the compound of the formula A3 and Mg and R5SSO₃M as defined herein; and
E) reacting the compounds provided in step A) or B) or C) or D) such as to obtain the liquid aromatic thioether.

As regards the definition of the compounds of formulae A, B, A1, C, D, HSR4, A3, HSR5, R5SSO₃M and preferred embodiments thereof, reference is made to the statements provided above when discussing the technical details of the liquid aromatic thioethers of the present invention.

In one embodiment, process step E) is carried out in a suitable solvent. Preferably, process step E) is carried out in an organic solvent. The term “organic solvent” does not exclude that the organic solvent comprises minor amounts of water. If the organic solvent comprises water, the organic solvent comprises water in an amount of from 0.01 to 10.0 wt.-%, preferably from 0.01 to 5.0 wt.-%, more preferably from 0.01 to 2.0 wt.-% and most preferably from 0.01 to 1.0 wt.-%, based on the total weight of the solvent. For example, the organic solvent is free of water.

Preferably, the solvent is selected such that the compounds provided in process step A), B), C) or D) are soluble in the solvent, such as the organic solvent, mixture of organic solvent and water, or water, preferably organic solvent.

The term “soluble” in the meaning of the present invention refers to systems in which no discrete solid particles of the compounds provided in process step A), B), C) or D) are observed in the solvent, such as organic solvent, mixture of organic solvent and water, or water, preferably organic solvent.

Preferably, the organic solvent is selected from the group comprising dichloromethane, propanol, dimethylformamide, ethanol, tetrahydrofuran, dimethylformamide, methanol, toluene, xylene, ethyl acetate and mixtures thereof.

In one embodiment, the organic solvent comprises, preferably consists of, a mixture of organic solvents, preferably two or three organic solvents, and most preferably two organic solvents.

Additionally or alternatively, process step E) is carried out at a temperature ranging from −20° C. to the reflux temperature of the organic solvent, preferably of the organic solvent in which process step E) is carried out. In one embodiment, process step E) is carried out at a temperature ranging from −20° C. to the reflux temperature of the organic solvent, preferably of the organic solvent in which process step E) is carried out. For example, process step C) is carried out at a temperature ranging from −20° C. to 100° C.

The amounts of the compounds of process step A), B), C) or D) in process step C) in order to reach the liquid armoatic thioether can vary in a broad range and can be determined by appropriate calculation.

In one embodiment, the process for the preparation of the liquid aromatic thioether further comprises a step F) of purifying the liquid aromatic thioether obtained in step E).

It is appreciated that step F) of purifying the liquid aromatic thioether obtained in step E) can be carried out by every means known to the skilled person for separating a compound from its reaction mixture. For example, process step F) can be carried out by methods selected from the group comprising, methods used for evaporating volatile compounds, such as in vacuum, extraction methods, distillation methods, chromatographic methods and mixtures thereof. Preferably, process step F) is carried out by evaporating volatile compounds, preferably in vacuum, extraction methods, distillation methods and chromatographic methods.

In one embodiment, the liquid aromatic thioethers are obtainable by the process for the preparation of the liquid aromatic thioether as defined herein.

The instant invention is thus further directed to a liquid aromatic thioether obtainable by the process for the preparation of a liquid aromatic thioether of the instant invention.

With regard to the definition of the liquid aromatic thioether and preferred embodiments thereof, reference is made to the statements provided above when discussing the technical details of the liquid aromatic thioether of the present invention.

In view of the advantages obtained, the present invention is further directed to an article comprising the liquid aromatic thioether as defined herein as an ingredient in at least one of its parts or as part of the article itself.

As regards the definition of the liquid aromatic thioether and preferred embodiments thereof, reference is made to the statements provided above when discussing the technical details of the liquid aromatic thioether of the present invention.

Preferably, the article comprising the liquid aromatic thioether is an optical lens such as a tuneable focus lens, optical liquid, wave guide material, tiltable prism, window material, color filter, coating, varnish, lacquer, dye or pigment formulation, immersion liquid, calibration liquid or matching liquid, ingredient or additive in a plastic material, ingredient or additive in a polymer.

In one embodiment, the article comprising the liquid aromatic thioether is an optical liquid.

In another embodiment, the article comprising the liquid aromatic thioether is a window material, color filter, coating, varnish, lacquer, dye or pigment formulation.

In another embodiment, the article comprising the liquid aromatic thioether is an immersion liquid, calibration liquid or matching liquid.

In another embodiment, the article comprising the liquid aromatic thioether is an ingredient or additive in a plastic material or an ingredient or additive in a polymer.

Furthermore, the present invention is directed to a use of the liquid aromatic thioether, as defined herein, as a component or substantial part of an optical liquid, window material, color filter, coating, varnish, lacquer, dye or pigment formulation, immersion liquid, calibration liquid or matching liquid, ingredient or additive in a plastic material or ingredient or additive in a polymer.

For example, the present invention is directed to the use of the liquid aromatic thioether, as defined herein, as optical liquid.

Further, the present invention is directed to the use of the liquid aromatic thioether, as defined herein, as window material, color filter, coating, varnish, lacquer, dye or pigment formulation.

Further, the present invention is directed to the use of the liquid aromatic thioether, as defined herein, as immersion liquid, calibration liquid or matching liquid.

Further, the present invention is directed to the use of the liquid aromatic thioether, as defined herein, as ingredient or additive in a plastic material or ingredient or additive in a polymer.

With regard to the definition of the liquid aromatic thioether and preferred embodiments thereof, reference is made to the statements provided above when discussing the technical details of the liquid aromatic thioether of the present invention.

The scope and interest of the invention will be better understood based on the following examples which are intended to illustrate certain embodiments of the invention and are non-limitative

### EXAMPLES

### 1. Methods

### NMR-Spectroscopy

NMR-data were acquired by using a VARIAN spectrometer Spectroscopin 300 at 300 K. The chemical shifts are given with respect to TMS as an internal standard δ-values (ppm). For the assignment of the signals and multiplicities the following abbreviations have been chosen: s—singulett, d—dublett, t—triplett, q—quartett, m—multiplett, b—broad, virt.—virtuell.

### Refractive Index n_{D} and Abbe's number ν_{D}

Refractive index and Abbe's number were measured at 25° C. with the digital nine-wavelength (approximately 404.7, 435.8, 486.1, 546.1, 587.56, 589.3, 632.8, 656.3 and 706.5 nm) refractometer DSR-λ of Schmidt & Haensch. The refractometer measures the critical angle of total reflection and calculates the refractive index from this value; the Abbe number (UD) is calculated by the instrument automatically. For measurements, 300 μL of the corresponding liquid collected by Eppendorf Research® plus 100-1 000 μL pipette were used. The refractive index n is reported at 589.3 nm.

### Gravity

Gravity has been defined as a mass of the 1cm³ (1 000 μL) of liquid being collected by Eppendorf Research® plus 100-1 000 μL pipette and weighed using the Kern Electronic Analytical Balance Type ABS 120-4N with readability of 0.1 mg and reproducibility of 0.2 mg.

### Viscosity

Dynamic shear viscosities of the synthesized liquids were measured in the cone-plate configuration with the Anton Paar MCR 301 rheometer operating in the rotational mode; the fixture CP50-0.5-SN20586 (diameter 50 mm and the angle of 0.5°) has been used as a measuring system; the gap size was set at 0.045 mm. Viscosities were measured as a continuous function of temperature (η−T)—within the temperature range from −20° C. to 100° C. The measurements were performed at two different shear rates: at 10 s⁻¹ and 100 s⁻¹.

### 2. Examples

### Example 1 (A2 Series)

### 2-[2-(3-bromophenyl)sulfanylethyl]-1,3-dithiolane

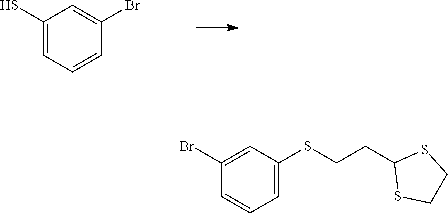 [SMILES: Sc1cccc(Br)c1 ; Brc1cccc(SCCC2SCCS2)c1]

3-Bromo-thiophenol (50.4 g, 0.287 mol) was dissolved in 145 ml dichloromethane (DCM) and cooled to −10° C. To this solution 25 ml (0.373 mol) acrolein were added slowly and after an additional 0.5 h at this temperature 6.0 ml (0.04 mol) triethylamine were added. The mixture was stirred for an additional 2 h until starting material is consumed according to a TLC. The mixture was diluted with 400 ml diethyl ether and subsequently extracted with sat. sodium hydrogen carbonate solution and brine. The organic layer was recovered, shortly stored over sodium sulfate and evaporated to give 70 g of an oil, which was immediately used in the ensuing step.

The oil was then dissolved in 250 ml DCM in an argon atmosphere and cooled to −10° C. Hereto 32.2 g (0.342 mol) 1,2-dithioethane dissolved in 50 ml DCM and 8.0 g of trifluoroborane etherate were added. The resulting mixture was stirred for 16 h at room temperature and extracted with water, 1 N sodium hydroxide solution and brine. Filtration of the organic phase over sodium sulfate yielded a slightly yellow oil, which was passed over a silica gel column (solvent: heptane-ethyl acetate: 50 -1). 44.4 g of the resulting transparent oil were further purified via distillation (220° C./0.076 mbar) to give 39.3 g of a transparent clear oil with a GC purity 99.4% and optical data:

n_{D}: 1.65846; Abbe's numbers ν: 27.8.

1H-NMR (300 MHz, CDCl₃) δ=7.47; (t, 1 H), 7.30; (ddd, 1 H), 7.24; (ddd, 1 H), 7.14; (t, 1H), 4.61; (t, 1H), 3.24; (s, 2H), 3.23; (s, 2H), 3.09-3.01; (m, 2H), 2.15-2.06; (m, 2H).

### Example 2 (A2 Series)

### 2-[2-(3-bromophenyl)sulfanyl-1-methyl-ethyl]-1,3-dithiolane

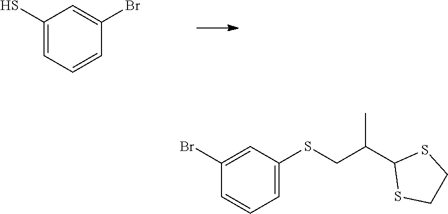 [SMILES: Sc1cccc(Br)c1 ; CC(CSc1cccc(Br)c1)C1SCCS1]

Example 2 was prepared in accordance with the description outlined for example 1 and using 0.816 g methacrolein and 2.005 g 3-bromo-thiophenol and subsequently 1.616 g of 1,2-dithioethane. 1.39 g of the title compound were obtained.

n_{D}: 1.64462; Abbe's numbers ν: 28.8.

1H-NMR (300 MHz, CDCl₃) δ=7.50; (t, 1H), 7.29; (ddd, 1 H), 7.26; (m, 1H), 7.13; (t, 1 H), 4.65; (d, 1H), 3.31; (dd, 1H), 3.22; (s, 2H), 3.21; (s, 2H), 2.79; (dd, 1H), 2.11-1.96; (m, 1H), 1.20; (d, 3H).

### Example 3 (A2 Series)

### 2-[2-(3-bromophenyl)sulfanylethyl]-2-methyl-1,3-dithiolane

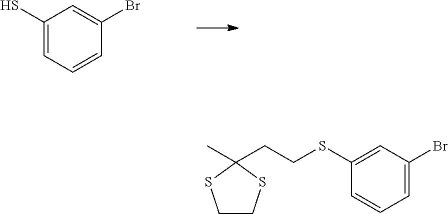 [SMILES: Sc1cccc(Br)c1 ; CC1(CCSc2cccc(Br)c2)SCCS1]

Example 3 was prepared in accordance with the description outlined for example 1 and using 0.940 g methylvinylketone and 2.002 g 3-bromo-thiophenol and subsequently 1.460 of 1,2-dithioethane. 2.41 g of the title compound were obtained.

n_{D}: 1.64101; Abbe's numbers ν: 28.6.

1H-NMR (300 MHz, CDCl₃) δ=7.49; (t, 1H), 7.28; (ddd, 1H), 7.25; (ddd, 1H), 7.14; (t, 1 H), 4.43-3.27; (m, 4H), 3.18-3.10; (m, 2H), 2.25-2.17; (m, 2H), 1.80; (s, 3H).

### Example 4 (A2 Series)

### 2-[2-[3-[2-(1,3-dithiolan-2-yl)ethylsulfanyl]phenyl]sulfanylethyl]-1,3-dithiolane

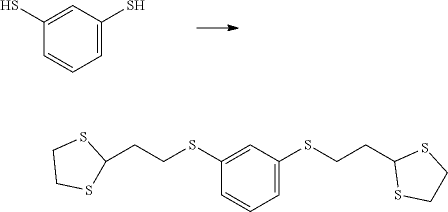 [SMILES: Sc1cccc(S)c1 ; c1cc(SCCC2SCCS2)cc(SCCC2SCCS2)c1]

1.008 g (7.09 mmol) of meta-dithiophenol were dissolved in 8 ml DCM and cooled to 0° C. in an argon atmosphere. Subsequently, 1.2 ml (16.1 mmol) of acrolein and 0.30 ml (2.15 mmol) of triethylamine were added and the mixture was stirred for 6.5 h at room temperature. The reaction mixture was then diluted with diethyl ether and subsequently extracted with sat. sodium hydrogen carbonate solution, water and brine. Filtration over sodium sulfate and evaporation gave 1.04 g of a yellow liquid which was dissolved together with 0.70 ml (8.35 mmol) 1,2-ethanediol in 15 ml DCM at 0° C. and treated with 0.10 ml (0.81 mmol) trifluoroborane etherate and then stirred for 6.5 hat room temperature. The mixture was subsequently extracted with water, 10% potassium hydroxide solution and brine. Filtration over silica gel yielded 0.81 g of the title compound as a transparent oil.

n_{D}: 1.69252; Abbe's numbers ν: 27.0.

1H-NMR (300 MHz, CDCl₃) δ=7.30; (t, 1H), 7.21-7.17; (m, 1H), 7.16-7.11; (m, 2H), 4.62; (t, 2H), 3.23; (d, 4H), 3.09-3.02; (m, 2H), 2.15-2.06; (m, 2H).

### Example 5 (A2 Series)

### 2-[2-[3-[2-(1,3-dithiolan-2-yl)propylsulfanyl]phenyl]sulfanyl-1-methyl-ethyl]-1,3-dithiolane

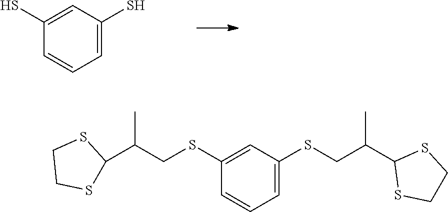 [SMILES: Sc1cccc(S)c1 ; CC(CSc1cccc(SCC(C)C2SCCS2)c1)C1SCCS1]

Example 5 was prepared in accordance with the description outlined for example 4 and using 2.70 g methacrolein and 3.00 g meta-dithiophenol and subsequently 0.580 of 1,2-dithioethane. 0.23 g of the title compound were obtained as viscous oil.

1H-NMR (300 MHz, CDCl₃) δ=7.34; (td, 1H), 7.2-7.11; (m, 3H), 4.67; (d, 2H), 3.29; (ddd, 2H), 3.20; (s, 4H), 2.80; (ddd, 2H), 2.11-1.96; (m, 2H), 1.19; (d, 6H).

### Example 6 (A1 Series)

### Methyl 3-[3-(3-methoxy-2-methyl-3-oxo-propyl)sulfanylphenyl]sulfanyl-2-methyl-propanoate

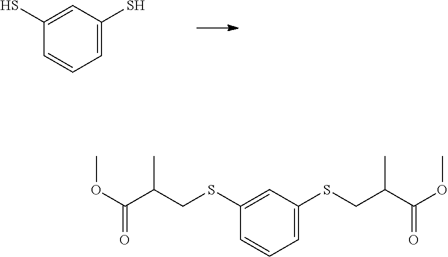 [SMILES: Sc1cccc(S)c1 ; COC(=O)C(C)CSc1cccc(SCC(C)C(=O)OC)c1]

Example 6 was prepared in accordance with the description outlined for example 4 and using 3.18 g methyl methacrylate and 10.58 g meta-dithiophenol in 19 ml of ethanol as solvent instead of DCM. 3.34 g of the title compound were obtained as oil.

n_{D}: 1.55322; Abbe's numbers ν: 31.2.

1H-NMR (400 MHz, CDCl₃) δ=7.33; (td, 1H), 7.23-7.15; (m, 3H), 3.67; (s, 6H), 3.26; (ddd, 2H), 2.93; (ddd, 2H), 2.70; (m, 2H), 1.27; (d, 6H).

### Example 7 (A1 Series)

### Tert-butyl 3-[3-(3-tert-butoxy-2-methyl-3-oxo-propyl)sulfanylphenyl]sulfanyl-2-methyl-propanoate

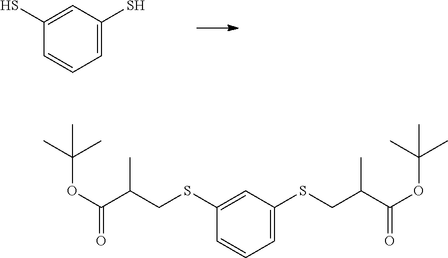 [SMILES: Sc1cccc(S)c1 ; CC(CSc1cccc(SCC(C)C(=O)OC(C)(C)C)c1)C(=O)OC(C)(C)C]

Example 7 was prepared in accordance with the description outlined for example 6 and using 3.98 g ^{t}butyl methacrylate and 1.00 g meta-dithiophenol in 13 ml of ethanol. 2.41 g of the title compound were obtained as oil.

n_{D}: 1.52107; Abbe's numbers ν: 34.4.

1H-NMR (300 MHz, CDCl₃) δ=7.33; (td, 1 H), 7.23-7.12; (m, 3H), 3.22; (dd, 2H), 2.88; (dd, 2H), 2.57; (m, 2H), 1.47; (s, 18H), 1.23; (d, 6H).

### Example 8 (A1 Series)

### 1,3-bis(2-diethoxyphosphorylethylsulfanyl)benzene

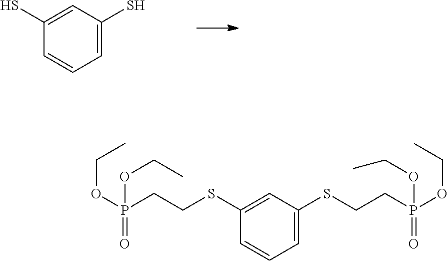 [SMILES: Sc1cccc(S)c1 ; CCOP(=O)(CCSc1cccc(SCCP(=O)(OCC)OCC)c1)OCC]

Example 8 was prepared in accordance with the description outlined for example 6 and using 2.54 g diethyl vinylphosphonate and 1.00 g meta-dithiophenol in 3 ml of ethanol. 2.24 g of the title compound were obtained as oil.

n_{D}: 1.51571; Abbe's numbers ν: 36.2.

1H-NMR (400 MHz, CDCl₃) δ=7.29; (t, 1H), 7.26-7.21; (m, 1H), 7.18-7.14; (m, 3H), 4.17-4.02; (m, 8H), 3.16-3.07; (m, 4H), 2.10-2.00; (m, 4H), 1.32; (t, 12H).

### Example 9 (A3 Series)

### 4-[(3-bromophenyl)sulfanylmethyl]-1,3-dithiolane

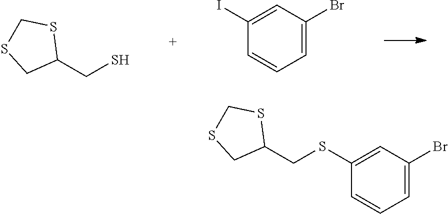

0.13 g (0.68 mmol) of copperiodide (Cul) and 3.749 g (27.1 mmol) of potassium carbonate (K₂CO₃) were suspended in an argon atmosphere in 13 ml ⁱpropanol containing 1.5 ml ethandiol cosolvent and 3.77 g (13.3 mmol) 1-bromo-3-iodobenzene and 2.06 g (13.5 mmol) of the reagent 1,3-dithiolan-4-ylmethanethiol. This suspension was heated to 90° C. for 28 h. After cooling down the mixture was filtered, the residues rinsed with cyclohexane and the combined organic phases subsequently extracted with satured sodium hydrogencarbonate and brine. Filtration over sodium sulfate and evaporation gave a yellow liquid, which was passed over silica gel (eluent: cyclohexane-toluene: 3-7) to yield 2.97 g of the title compound as colorless liquid.

n_{D}: 1.67717; Abbe's numbers ν: 26.8.

1 H-NMR (300 MHz, CDCl₃) δ=7.51; (t, 1H), 7.34; (ddd, 1 H), 7.29; (ddd, 1 H), 7.16; (t, 1 H), 3.95; (d, 1 H), 3.90; (d, 1 H), 3.82; (m, 1 H), 3.35; (dd, 1 H), 3.23-3.09; (m, 3H).

### Example 10 (A3 Series)

### 1 -Bromo-3-(2-methyl butylsulfanyl)benzene

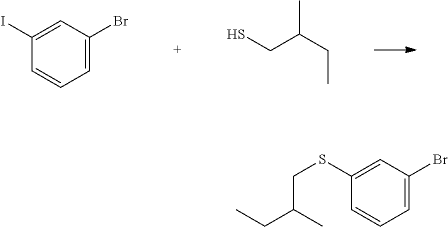

Example 10 was prepared in accordance with the description outlined for example 9 and using 11.10 g 1-bromo-3-iodobenzene and 4.14 g 2-methylbutanethiol. 8.54 g of the title compound were obtained as liquid.

n_{D}: 1.5590; Abbe's numbers ν: n.d.

1 H-NMR (300 MHz, CDCl₃) δ=7.42; (t, 1H), 7.24; (ddd, 1 H), 7.20; (ddd, 1H), 7.08; (t, 1H), 2.92; (dd, 1H), 2.73; (dd, 1H), 1.75-1.59; (m, 1H), 1.58-1.45; (m, 1H), 1.35-1.19; (m, 1H), 0.91; (t, 3H).

### Example 11 (A3 Series)

### 1 -Bromo-3-cyclohexylsulfanyl-benzene

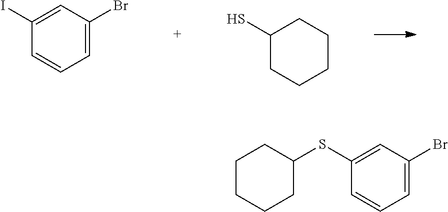 [SMILES: Brc1cccc(I)c1 ; SC1CCCCC1 ; Brc1cccc(SC2CCCCC2)c1]

Example 11 was prepared in accordance with the description outlined for example 9 and using 10.21 g 1-bromo-3-iodobenzene and 4.66 g cyclohexanethiol. 8.46 g of the title compound were obtained as liquid.

n_{D}: 1.595; Abbe's numbers ν: n.d.

1 H-NMR (300 MHz, CDCl₃) δ=7.54; (t, 1 H), 7.31; (ddd, 1H), 7.29; (ddd, 1 H), 7.11; (t, 1H), 3.13; (dt, 1H), 2.03-1.95; (m, 2H), 1.82-1.72; (m, 2H), 1.66-1.57; (m, 1H), 1.46-1.20; (m, 5H).

### Example 12 (A4 Series)

### 4-[[3-(1,3-dithiolan-4-ylmethylsulfanyl)phenyl]sulfanylmethyl]-1,3-dithiolane

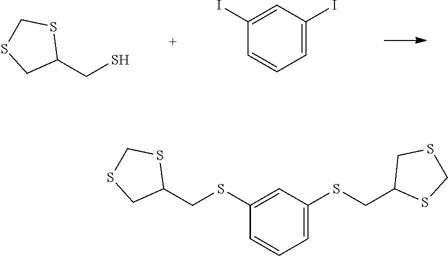 [SMILES: SCC1CSCS1 ; Ic1cccc(I)c1 ; c1cc(SCC2CSCS2)cc(SCC2CSCS2)c1]

9.59 g (29.1 mmol) 1,3-diiodobenzene, 0.83 g (4.37 mmol) copperidodide and 16.06 g (116.20 mmol) potassiumcarbonate were dispersed in a mixture of 55 ml ⁱpropanol and 6.5 ml 1,2-ethandiol in an argon atmosphere. After addition of 9.10 g (59.78 mmol) of 1,3-dithiolan-4-ylmethanethiol the suspension was heated at 90° C. for 24 h. After cooling down, the suspension was filtered and the organic phase diluted with ethyl acetate and subsequently extracted with sat. sodium carbonate solution and brine. Filtration over sodium sulfate and evaporation gave a yellow liquid, which was purified over silica gel (eluent: cyclohexane-toluene: 3-7) to yield 4.01 g of the title compound as colorless liquid.

n_{D}: 1.69252; Abbe's numbers ν: 27.0.

1H-NMR (300 MHz, CDCl₃) δ=7.37; (t, 1H), 7.24-7.17; (m, 3H), 3.94; (d, 2H), 3.90; (d, 2H), 3.86-3.77; (m, 2H), 3.55; (dd, 2H), 3.23-3.09; (m, 6H).

### Example 13 (A4 Series)

### 1,3-bis(cyclohexylsulfanyl)benzene

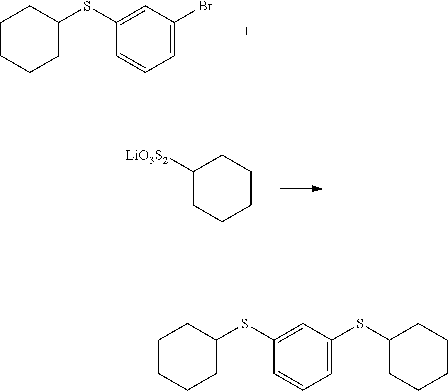

0.810 g (33.32 mmol) of magnesium turnings were suspended in 25 ml of dry tetrahydrofuran in an argon atmosphere. Then, 8.00 g (24.52 mmol) of 1-bromo-3-cyclohexyl-sulfanyl-benzene were slowly added with vigorous stirring and the mixture refluxed for 6 h. After cooling down to 0° C., 5.00g (15.07 mmol) of the Bunte salt (lithium salt of cycolhexyl thiosulfate) were added and the mixture further diluted with 20 ml tetrahydrofuran. Stirring the resulting suspension was continued for another 17 h. The mixture was then filtered and extracted with satured ammonium chloride and satured sodium hydrogen carbonate. Filtration over sodium sulfate and evaporation of the solvent gave an oil which was purified over silica gel (eluent: cyclohexane) to yield 3.45 g of the title compound.

n_{D}: 1.58160; Abbe's numbers ν: 31.3.

1H-NMR (300 MHz, CDCl₃) δ=7.41; (t, 1H), 7.25-7.15; (m, 3H), 3.11; (tt, 2H), 2.06-1.91; (m, 4H), 1.83-1.72; (m, 4H), 1.66-1.57; (m, 2H), 1.47-1.17; (m, 10H).

### Example 14 (A4 Series)

### 1,3-bis(2-methylbutylsulfanyl)benzene

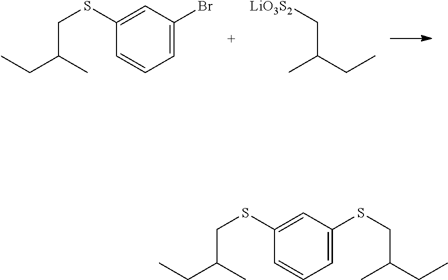

Example 14 was prepared in accordance with the description outlined for example 13 and using 8.15 g 1-bromo-3-(2-methylbutyl-sulfanyl)benzene and 10.41 g of the Bunte salt (lithium salt of cycolhexyl thiosulfate). 4.38 g of the title compound were obtained as colorless liquid.

n_{D}: 1.55067; Abbe's numbers ν: 30.4.

1 H-NMR (400 MHz, CDCl₃) δ=7.25; (t, 1 H), 7.16-7.11; (m, 1H), 7.07-7.05; (m, 2H), 2.92; (dd, 2H), 2.73; (dd, 2H), 1.72-1.59; (m, 2H), 1.59-1.46; (m, 2H), 1.34-1.18; (m, 2H), 1.01; (d, 6H), 0.90; (t, 6H).

### Example 15 (A4 Series)

### 1 -cyclohexylsulfanyl-3-(2-methylbutylsulfanyl)benzene

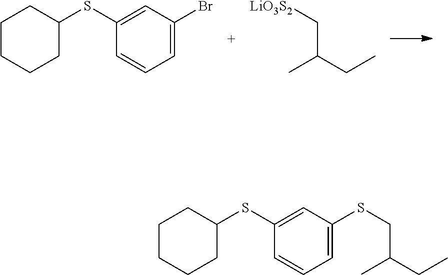

Example 15 was prepared in accordance with the description outlined for example 13 and using 4.50 g 1-bromo-3-cyclohexyl-sulfanyl-benzene and 4.00 g of the Bunte salt (lithium salt of cycolhexyl thiosulfate). 1.90 g of the title compound were obtained as colorless liquid.

n_{D}: 1.56649; Abbe's numbers ν: 30.8.

1H-NMR (400 MHz, CDCl₃) δ=7.34-7.32; (m, 1H), 7.20-7.13; (m, 3H), 3.11; (tt, 1H), 2.94; (dd, 1H), 2.74; (dd, 1 H), 2.03-1.94; (m, 2H), 1.81-1.74; (m, 2H), 1.71-1.58; (m, 2H), 1.58-1.48; (m, 1H), 1.41-1.19; (m, 6H), 1.02; (d, 3H), 0.91; (t, 3H).

1. A liquid aromatic thioether, satisfying formula A1, A2, A3, or A4: 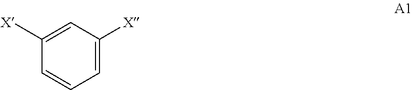
wherein X′=Cl, Br, I, or X″, and
X″=S(CH₂CHR1)-Z,
wherein Z=C(O)G, C(O)A-G, or P(O)(OR2)₂,
wherein G=H, Me, Et, ⁱProp, ⁿProp, or ^{t}But,
A=O or NH,
R1=H or Me, and
R2=Me, Et, ⁿProp, or ⁱProp; 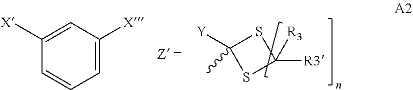
wherein X′=Cl, Br, I, or X″′, and
X″′=S(CH₂CHR1)-Z′,
wherein Z′ is as defined above,
wherein each R1, R3, and R3′ is independently H or Me,
n=2 or 3, and
Y=H, Me, Et, ⁱProp, ⁿProp, or ^{t}But; 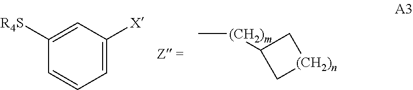
wherein X′=I or Br, and
R4=a C1 to C8 linear or branched alkyl chain, a carbocycle with 5 or 6 C-atoms, or Z″ as defined above, wherein m and n are each independently 1, 2, or 3 and wherein one or two non-neighbouring ring-forming —CH₂-moieties of Z″ are replaced by sulfur; and 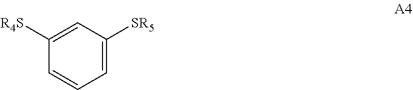
wherein R4 and R5 are each independently a C1 to C8 linear or branched alkyl chain, a carbocycle with 5 or 6 C-atoms, or Z″ as defined above.

2. The liquid aromatic thioether of claim 1, which satisfies formula A1 and is a reaction product of compounds of the following formulae A and B: 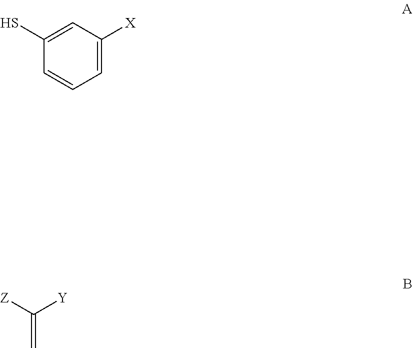
wherein, in formula A, X=Cl, Br, I, or SH; and
in formula B, Y=H, Me, Et, ⁱProp, ⁿProp, or ^{t}But, and
Z=C(O)G, C(O)A-G, or P(O)(OR2)₂,
wherein G=H, Me, Et, ⁱProp, ⁿProp, or ^{t}But,
A=O or NH, and
R2=Me, Et, ⁿProp, or ⁱProp.

3. (canceled)

4. The liquid aromatic thioether of claim 1, which satisfies formula A2 and is a reaction product of compounds of the following formulae A1 and C: 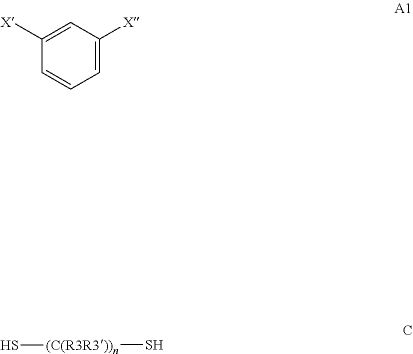
wherein, in formula A1, X′=Cl, Br, I, or X″, and
X″=S(CH₂CHR1)-Z
wherein Z=C(O)G, G=H, Me, Et, ⁱProp, ⁿProp, or ^{t}But, and
R1=H or Me; and
in formula C, n=2 or 3, and
R3 and R3′ are each independently H or Me.

5. (canceled)

6. The liquid aromatic thioether of claim 1, which satisfies formula A3 and is a reaction product of a compound of the following formula D and an alkylthiol HSR4: 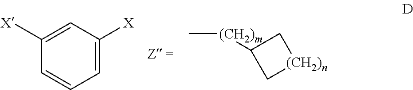
wherein, in formula D, X=I, and
X′=Br or X=X′=I or Br; and
in the alkylthiol, R4=a C1 to C8 linear or branched alkyl chain, a carbocycle with 5 or 6 C-atoms, or Z″ as defined above, wherein m and n are each independently 1, 2, or 3 and wherein one or two non-neighbouring ring-forming —CH₂-moieties of Z″ are replaced by sulfur.

7. (canceled)

8. The liquid aromatic thioether of claim 1, which satisfies formula A4 and is a reaction product of a compound of the following formula D and an alkylthiol HSR5: 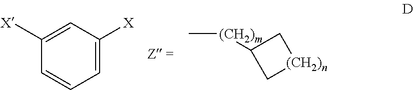
wherein, in formula D, X=I, and
X′=Br or X=X′=I or Br; and
in the alkylthiol HSR5, R5=a C1 to C8 linear or branched alkyl chain, a carbocycle with 5 or 6 C-atoms, or Z″ as defined above, wherein m and n are each independently 1, 2, or 3 and wherein one or two non-neighbouring ring-forming —CH₂-moieties of Z″ are replaced by sulfur, wherein and
R4 and R5 are the same or different.

9. The liquid aromatic thioether of claim 1, which satisfies formula A4 and is a reaction product of a compound of the following formula A3 and Mg, wherein the reaction product is a Grignard reagent of formula A3, 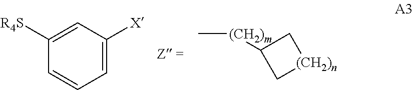
wherein, in formula A3, X′=I or Br, and
R4=a C1 to C8 linear or branched alkyl chain, a carbocycle with 5 or 6 C-atoms, or Z″ as defined above, wherein m and n are each independently 1, 2, or 3 and wherein one or two non-neighbouring ring-forming —CH₂-moieties of Z″ are replaced by sulfur; or
which satisfies formula A4 and is a reaction product of the Grignard reagent of A3 and R5SSO₃M,
wherein M=Li or Na, and
R5=a C1 to C8 linear or branched alkyl chain, a carbocycle with 5 or 6 C-atoms or Z″ as defined above, wherein m and n are each independently 1, 2, or 3 and wherein one or two non-neighbouring ring-forming —CH₂-moieties of Z″ are replaced by sulfur, wherein and
R4 and R5 are the same or different.

10. The liquid aromatic thioether of claim 1, which has a refractive index in a range of from 1.50 to 1.9 and/or an Abbe's number in a range of from 25 to 110.

11. A process for preparing the liquid aromatic thioether of claim 1, the process comprising:
A) providing compounds of the following formulae A and B: 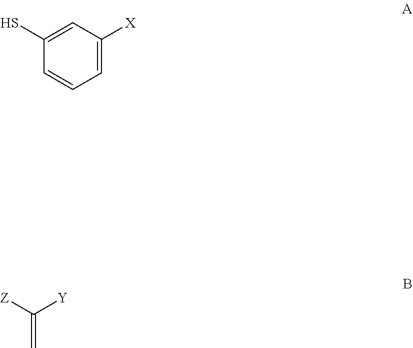
wherein, in formula A, X=Cl, Br, I, or SH, and
in formula B, Y=H, Me, Et, ⁱProp, ⁿProp, or ^{t}But, and
Z=C(O)G, C(O)A-G, or P(O)(OR2)₂.
wherein G=H, Me, Et, ⁱProp, ⁿProp, or ^{t}But,
A=O or NH; or
B) providing compounds of the following formulae A1 and C: 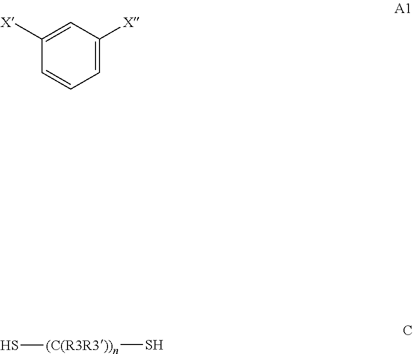
wherein, in formula A1, X′=Cl, Br, I, or X″, and
X″=S(CH₂CHR1)-Z
wherein Z=C(O)G, G=H, Me, Et, ⁱProp, ⁿProp, or ^{t}But, and
R1=H or Me, and
in formula C, n=2 or 3, and
R3 and R3′ are each independently H or Me; or
C) providing a compound of the following formula D and an alkylthiol HSR4: 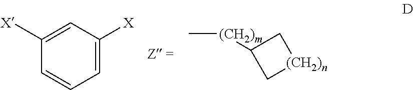
wherein, in formula D, X=I, and
X′=Br or X=X′=I or Br; and
in the alkylthiol, R4=a C1 to C8 linear or branched alkyl chain, a carbocycle with 5 or 6 C-atoms, or Z″ as defined above, wherein m and n are each independently 1, 2, or 3 and wherein one or two non-neighbouring ring-forming —CH₂moieties of Z″ are replaced by sulfur; or
D) providing a compound of above formula D and an alkylthiol HSR5 or a compound of the following formula A3 and Mg and an alkylthiol R5SSO₃M: 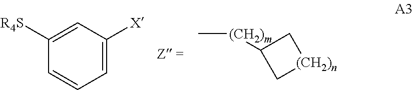
wherein, in formula A3, X′=I or Br, and
R4=a C1 to C8 linear or branched alkyl chain, a carbocycle with 5 or 6 C-atoms, or Z″ as defined above, wherein m and n are each independently 1, 2, or 3 and wherein one or two non-neighbouring ring-forming —CH₂-moieties of Z″ are replaced by sulfur, and
M=Li or Na, and
in the alkylthiols HSR5 and R5SSO₃M, each R5 is independently a C1 to C8 linear or branched alkyl chain, a carbocycle with 5 or 6 C-atoms or Z″ as defined above, wherein in and n are each independently 1, 2, or 3 and wherein one or two non-neighbouring ring-forming —CH₂-moieties of Z″ are replaced by sulfur, and
R4 and R5 are the same or different; and
E) reacting the compounds provided in A), B), C), or D), to obtain the liquid aromatic thioether.

12. An optical lens, an optical liquid, a wave guide material, a tiltable prism, a window material, a color filter, a coating, a varnish, a lacquer, a dye or pigment formulation, an immersion liquid, a calibration or matching liquid, an ingredient or additive in a plastic material, or an ingredient or additive in a polymer, comprising the liquid aromatic thioether of claim 1 as a part or as an ingredient in at least one of its parts,
wherein the optical lens may be a tuneable focus lens.

13. (canceled)

14. The liquid aromatic thioether of claim 1, which satisfies formula A1.

15. The liquid aromatic thioether of claim 1, which satisfies formula A2.

16. The liquid aromatic thioether of claim 1, which satisfies formula A3.

17. The liquid aromatic thioether of claim 1, which satisfies formula A4.
